(19) United States
(12) Patent Application Publication
(10) Pub. No.: US 2010/0262047 A1
(43) Pub. Date: Oct. 14, 2010

(54) PHYSICAL THERAPY SYSTEMS AND METHODS

(75) Inventor: Vladimir Genis, Warminster, PA (US)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(73) Assignee: DREXEL UNIVERSITY, Philadelphia, PA (US)

(21) Appl. No.: 12/725,239

(22) Filed: Mar. 16, 2010

Related U.S. Application Data
(60) Provisional application No. 61/167,679, filed on Apr. 8, 2009.

(51) Int. Cl. A61H 1/00; A41D 19/00; A61B 5/00

(52) U.S. Cl. 601/5; 2/159; 600/309

(57) ABSTRACT

The invention is directed to a physical therapy system including a glove with a set of acoustic sensors and a positive feedback system and method for teaching finger extension. The system may also include a pair of gloves with a set of acoustic sensors and a positive feedback system to teach clapping. In another aspect, the system may include a glove with an acoustic sensor, an object with a corresponding sensor and a positive feedback system for teaching arm extension. The invention may be particularly useful for providing a rehabilitation method for individuals affected with hemiplegic cerebral palsy.

### BACKGROUND OF THE INVENTION

1. Field of the Invention

The invention is directed to therapeutic systems and methods for rehabilitating disabled or injured limbs. The invention may be particularly useful as a rehabilitation system for children diagnosed with hemiplegic cerebral palsy.

2. Description of the Related Technology

Traditional constraint-induced movement therapy has recently demonstrated promising results in rehabilitating the disabled limbs of children diagnosed with hemiplegia. Constraint-induced therapy generally involves restraining a child's unaffected or less affected arm. Under the guidance of a physical therapist, the child is then forced to perform structured behavioral reinforcement exercises using his disabled arm, which is typically flexed at the elbow and characterized by a clenched fist. The extent of rehabilitation is dependent solely on the behavioral reinforcement exercises. Current behavioral reinforcement exercises are limited in their range of motion and do not provide comprehensive rehabilitation of the affected arm. Additionally, traditional constraint-induced movement therapy requires the presence of a physical therapist to ensure that the exercises are properly executed and provide positive feedback. Furthermore, only older children, at least 1 year old, who have already learned to use their affected limb to manipulate objects are eligible for this type of therapy.

Automated physical therapy devices and methods have also been developed to facilitate the rehabilitation of individuals afflicted with a wide range of illnesses. These devices, however, are poorly designed. Inefficient and low sensitivity sensors that are inadequately arranged provide unreliable results. Consequently, these systems are disposed to producing false positive and false negative results.

For example, U.S. Pat. No. 6,360,615 (Smela) discloses a fabric constructed from a conductive piezoelectric material that may used to rehabilitate a limb. The device may be sensitive to small movement and may facilitate rehabilitation by providing an audible or visual feedback signal when a particular motion is achieved. Smela describes an application wherein the conducting polymer coated fabric covers a finger. When the finger is bent, the resistance of the fabric changes as the fabric is stretched. A circuit detects the change in resistance and induces a positive feedback, such as an audible or visual signal. This system, however, is primitive in that the mechanically sensitive piezoelectric sensors are inclined to produce false positive responses. Moreover, Smela does not teach a physical therapy method for individuals afflicted with hemiplegic cerebral palsy.

Similarly, “Physical Therapy Device for Infants with Hemiplegia,” discloses a linear variable differential transformer (LVDT) sensor attached to a glove for training and measuring finger displacement. The position and displacement of the LVDT sensor was measured over time and correlated to a calibration curve to approximately determine finger motion and position. Because this system can only approximate finger position and because the calibration curve is not reliably accurate for all users, there is a need to develop a more accurate and effective system.

Another device disclosed in “Senior Design Project in Biomedical Engineering Education” discloses a system including a glove and wall board for training and measuring arm displacement using proximity sensors. A small metallic object was embedded in the glove, and a corresponding proximity sensor was embedded in the wall board to detect the distance of the glove using a change in magnetic induction. Because the magnetic field cannot be focused in a specific direction, there is a need to develop a more accurate sensor and measurement system.

Other physical therapy systems, such as that disclosed in U.S. Pat. No. 7,481,782 (Scott), involve rehabilitation devices that induce movement of a limb rather than merely monitoring a patient's movement and providing positive feedback. Scott, for example, discloses a glove with an electromechanical actuator with a piezoelectric polyvinylidene fluoride (PVDF) membrane. Although this system induces finger movement, it is not designed to monitor voluntary finger displacement.

Therefore there is a need to develop an effective and highly accurate system and method for rehabilitating the disabled limbs of individuals suffering from hemiplegic cerebral palsy.

### SUMMARY OF THE INVENTION

The invention relates to a novel system and a physical therapy method for rehabilitating the affected limbs of individuals such as those diagnosed with hemiplegic cerebral palsy.

In a first aspect, the invention is directed to a physical therapy system including a glove; a first piezoelectric sensor capable of transmitting an acoustic signal; a second piezoelectric sensor capable of receiving the acoustic signal transmitted by the first piezoelectric sensor, wherein each of the first and second piezoelectric sensors is operatively associated with the glove in a manner whereby the acoustic signal transmitted by the first piezoelectric sensor may be received by the second piezoelectric sensor and converted to an electrical signal; and a positive feedback system operably associated with the second piezoelectric sensor to provide positive feedback responsive to the electrical signal of said second sensor.

In a second aspect, the invention is directed to a physical therapy system including first and second objects; a first piezoelectric sensor capable of transmitting an acoustic signal; a second piezoelectric sensor capable of receiving the acoustic signal transmitted by the first piezoelectric sensor, and wherein one said piezoelectric sensor is operatively associated with the first object and one said piezoelectric sensor is operatively associated with the second object in a manner whereby the acoustic signal transmitted by the first piezoelectric sensor may be received by the second piezoelectric sensor and converted to an electrical signal; and a positive feedback system operably associated with the second piezoelectric sensor to provide positive feedback responsive to said electrical signal from said second piezoelectric sensor.

In an third aspect, the invention is directed to a physical therapy method that involves providing a physical therapy system including a glove having first and second piezoelectric sensors operatively associated therewith in a manner whereby an acoustic signal transmitted by the first piezoelectric sensor may be received by the second piezoelectric sensor, and a positive feedback system operatively associated with the second piezoelectric sensor; placing the glove on a hand of an individual; and moving the hand until a predetermined condition is met, to thereby activate the positive feedback system.

In a fourth aspect, the invention is directed to a physical therapy method that involves providing a physical therapy system including first and second objects. A first piezoelectric sensor adapted to transmit an acoustic signal is operatively associated with one of said first an second objects and a second piezoelectric sensor adapted to receive an acoustic signal operatively associated with another of said first and second objects in a manner whereby said second piezoelectric sensor may receive the signal transmitted by the first piezoelectric sensor and converts the acoustic signal to an electrical signal, and a positive feedback system operatively connected to said second piezoelectric sensor responsive to said electrical signal from said second piezoelectric sensor.

### BRIEF DESCRIPTION OF THE DRAWINGS

FIG. 1(a) shows an exemplary physical therapy system for training finger extension.

FIG. 1(b) shows a schematic circuit diagram for the system of FIG. 1(a).

FIG. 2(a) is schematic operational diagram of FIG. 1(a) wherein the system circuitry is turned off when the fingers of the glove are in an initial clenched position.

FIG. 2(b) is schematic operational diagram of FIG. 1(a) wherein the system circuitry is turned on when the fingers of the glove are extended.

FIG. 2(c) is flow chart showing the operation of the embodiment of FIG. 1(a).

FIG. 3 shows an exemplary physical therapy system for training an individual to clap.

FIG. 4 is flow chart showing the operation of the embodiment of FIG. 3.

FIG. 5(a) shows a glove and object of a physical therapy system for training arm extension.

FIG. 5(b) shows another embodiment of a physical therapy system for training arm extension which is activated by the user sitting on a seat cushion.

FIG. 6 is flow chart showing the operation of the embodiment of FIG. 5(b).

### DETAILED DESCRIPTION OF THE PREFERRED EMBODIMENTS

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in other systems and methods. Before explaining the disclosed embodiments of the present invention in detail, it is to be understood that the invention is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Thus, for example, reference to “a sensor” may include a plurality of sensors and equivalents thereof known to those skilled in the art, and so forth. As well, the terms “a” (or “an”), “one or more” and “at least one” can be used interchangeably herein. It is also to be noted that the terms “comprising”, “including”, “composed of and “having” can be used interchangeably.

The present invention is directed to novel physical therapy systems and methods for rehabilitating limbs. Specifically, the invention may be used to train finger extension, hand clapping and arm extension. It is envisioned that the physical therapy systems and methods of the invention may be particularly useful for rehabilitating the impaired limbs, such as arms and fingers, of individuals diagnosed with hemiplegic cerebral palsy. The invention may be useful for rehabilitating any disabled, deformed or injured limbs. In an exemplary embodiment, the invention may be useful for rehabilitating the limbs of wounded soldiers or individuals who have sustained bone fractures.

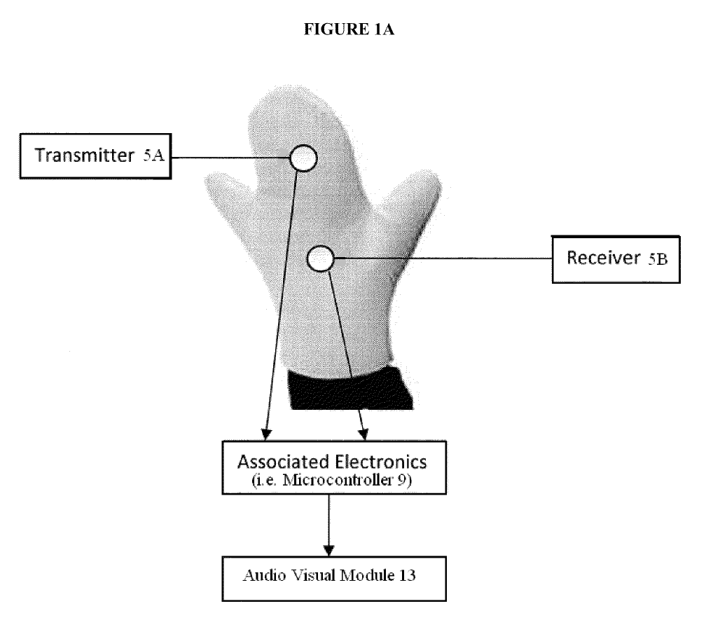

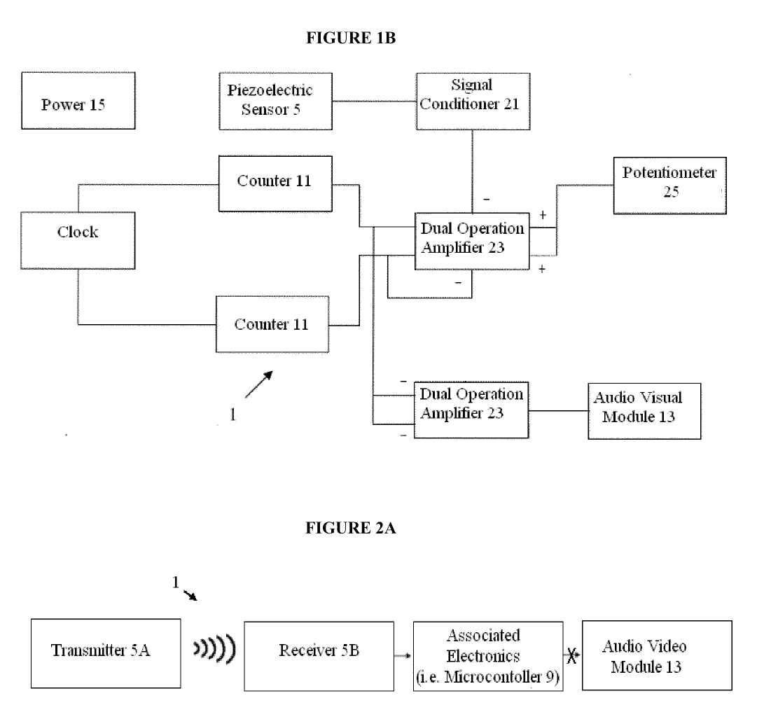

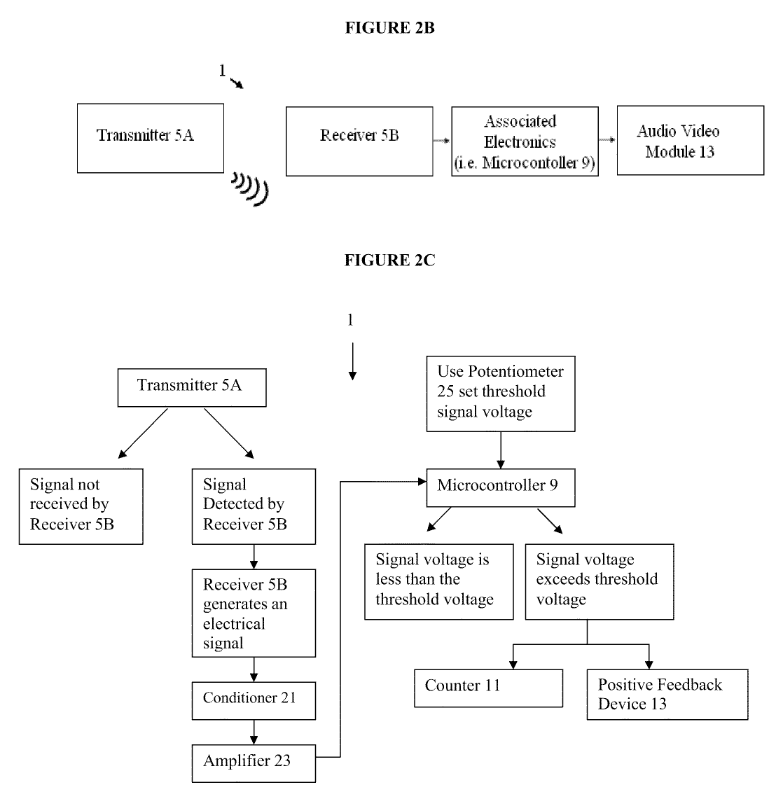

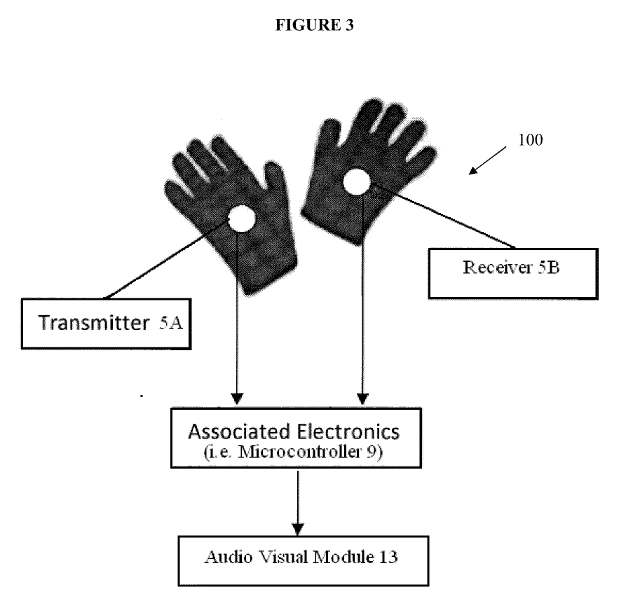

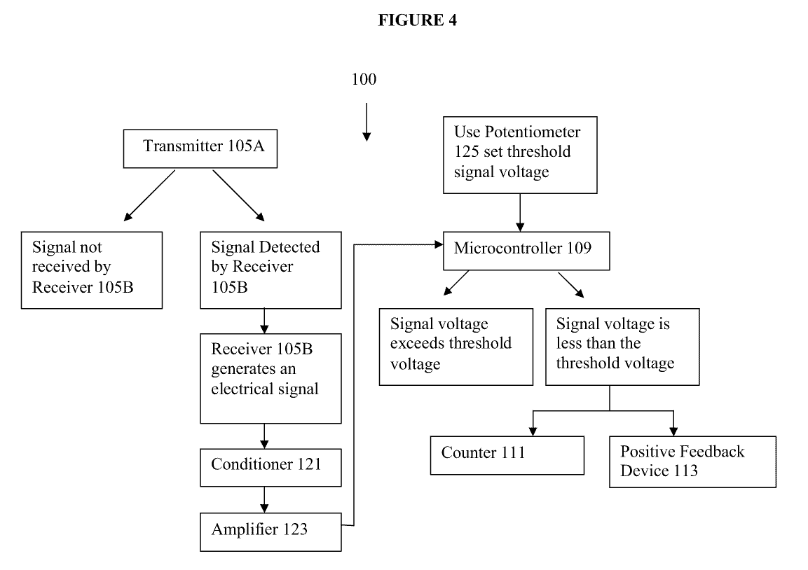

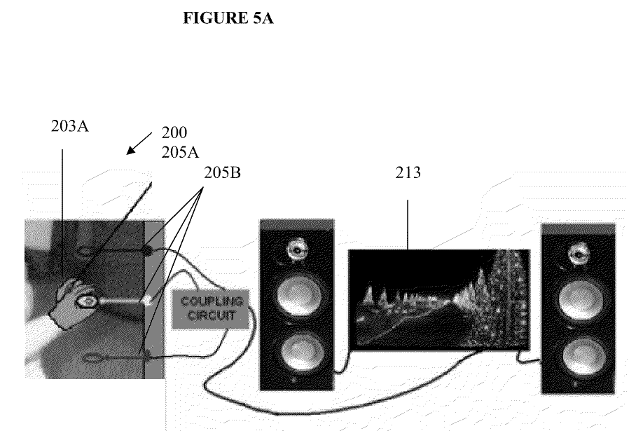

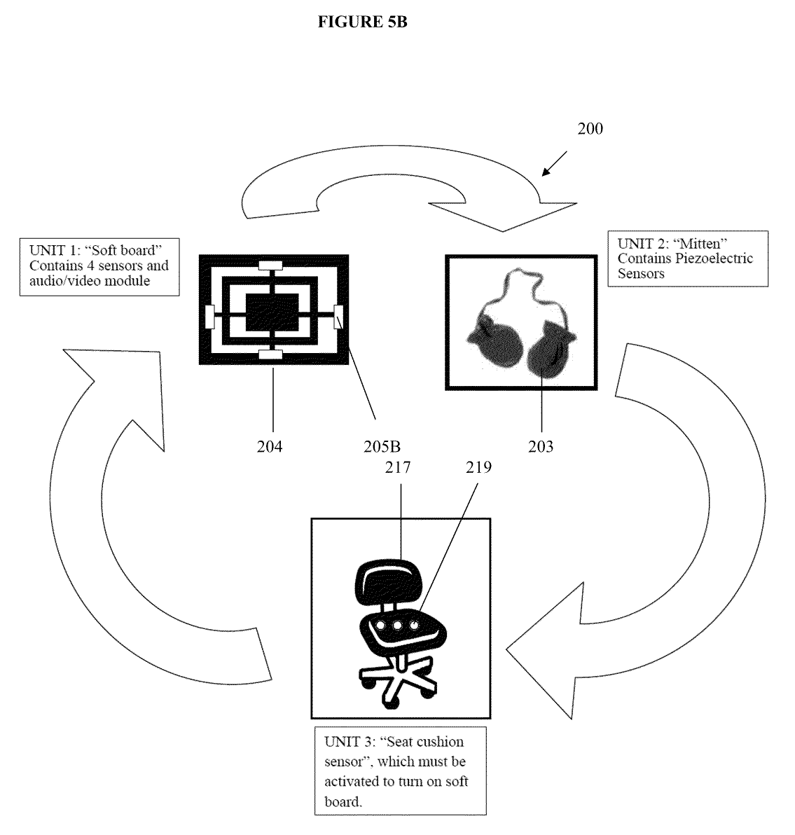

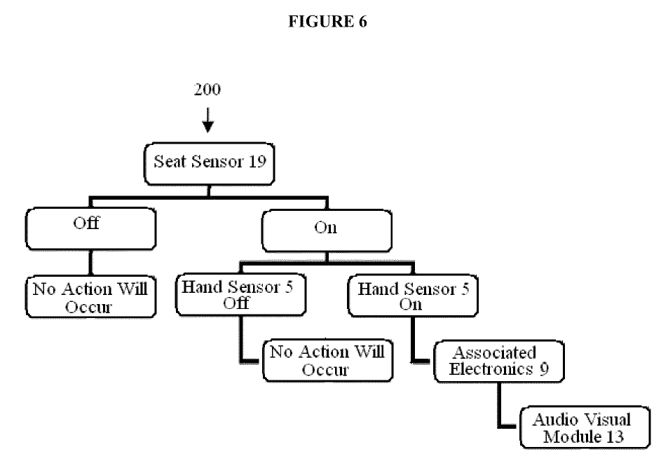

In a first exemplary embodiment shown in FIGS. 1-2, a physical therapy system for teaching and monitoring finger extension includes a positive feedback system and a glove having a pair of sensors for transmitting and receiving acoustic signals. In a second exemplary embodiment shown in FIGS. 3-4, a physical therapy system for teaching and monitoring clapping includes a positive feedback system and a pair of gloves, wherein one glove includes a transmitter and the second glove includes a receiver. In a third exemplary embodiment shown in FIGS. 5-6, a physical therapy system for teaching and monitoring arm extension includes a glove with a sensor capable of transmitting or receiving an acoustic signal and an object spaced apart from the glove having a receiver.

FIGS. 1(a)-(b) show an exemplary embodiment of a first physical therapy system 1 for training finger extension. The system is constructed as a glove 3 provided with at least two sensors 5A, 5B. The sensors 5A, 5B include at least one sensor 5A capable of transmitting an acoustic signal and at least one sensor 5B capable of receiving an acoustic signal. System 1 also includes a positive feedback system 7.

Gloves 3 may have any shape, size, configuration or material composition suitable for accommodating children and adults of varying ages. Alternatively, gloves 3 may be replaced by any device which performs the function of associating sensors 5A, 5B with a body part of the user at a particular location and, if necessary, in the desired orientation. Thus, alternative devices may include mittens, fingerless gloves, gloves with one or more fingers, devices which fit only over one or more fingers, etc. The important aspect is that for each system, the gloves 3 or alternative devices may be specially designed to position sensors 5A, 5B in the desired position and, if necessary, orientation, relative to one another such that a property of the acoustic signal received by the receiving sensor 5B can be correlated to a predetermined condition of the user's body part. Typically, the predetermined condition will correspond to a particular movement or series of movements of the body part for which training is desired.

In an exemplary embodiment, glove 3 may be constructed from fire retardant or electrically insulating materials, such as neoprene. Glove 3 may also be optionally water resistant, corrosion resistant, non-conducting, non-irritating, constructed from allergen-free materials or any combination thereof. An exemplary material may be cotton. The weight and size of glove 3 should not substantially hinder the natural movement of a user's hand unless the particular training exercise requires positive or negative reinforcement, in which case glove 3 can be specially designed for such purpose. Preferably, glove 3 may weigh about 25% by weight of the user's hand or less. In an exemplary embodiment, glove 3 may weigh about 10 g or less.

Sensors 5A, 5B may be operatively associated with glove 3. Sensors 5A, 5B may be constructed as at least one transmitter 5A and at least one receiver 5B. For example, sensors 5A, 5B may be positioned on a surface of, within the material of, and/or in an interior cavity of glove 3 in order to accurately detect and monitor finger motion, particularly finger extension. Sensors 5, and any electronic components or connections of system 1 are preferably positioned so as to avoid contact with a user's skin when the device is in use.

One or more transmitters 5A may be positioned at a distal region of glove 3 corresponding to the approximate location of one or more of an individual's fingers. In one embodiment, one or more transmitters 5A may be positioned on a surface of the glove 3 which faces the same direction as the palm of the glove 3 when the glove is located on the hand of an intended wearer. Transmitter 5A may be located anywhere along the portion of glove 3 housing an individual's fingers, including any region of the glove corresponding to the lower, middle or upper phalanges of the fingers when the glove is located on the hand of an intended wearer. In an exemplary embodiment, one or more transmitters 5A may span the length of one or more phalanges. In another embodiment, one or more transmitters 5A may be positioned on each finger, including or excluding the thumb. In another embodiment, when glove 3 is constructed as a mitten, one or more transmitters 5A may span the width of one or more fingertips of the mitten, preferably the entire width of the distal end of the mitten.

One or more receivers 5B may be positioned at a proximal region of glove 3 that corresponds to any position along an individual's palm, including the base, middle or upper region of the palm, when the glove is worn by an intended wearer. Alternatively, receivers 5B may be located at a position corresponding to the base of the fingers when the glove is worn by an intended wearer. In an exemplary embodiment, one or more receivers 5B may span the width of the palm or one or more finger bases when the glove is worn by an intended wearer. One receiver 5B may be operatively associated with and receive acoustic signals from two or more, a plurality of or all transmitters 5A positioned on glove 3. Alternatively, each receiver 5B may be operatively associated with only one transmitter 5A so that there is a unique corresponding receiver 5B for each transmitter 5A.

Sensors 5A, 5B of the physical therapy system 1 may be any suitable sensor capable of transmitting or receiving acoustic signals. In an exemplary embodiment, sensor 5A, 5B may be any piezoelectric sensor capable of transmitting or receiving acoustic signals, preferably ultrasonic signals. Preferably, sensors 5A, 5B operates in a frequency range of about 20 kHz to 200 kHz and the intensity of the signal may be less than about 100 mW/cm². Exemplary sensors 5A, 5B may be constructed from polyvinylidene fluoride (PVDF). Sensors 5A, 5B may also be connected to a power supply, such as an outlet or a portable power source, such as a battery, using conventional means.

Transmitter 5A is preferably capable of transmitting an acoustic signal in a specific direction, which signal may or may not be focused. In an exemplary embodiment, transmitter 5A is positioned at the finger region of glove 3 and transmits an acoustic signal. In an exemplary embodiment, the transmission may be focused to provide greater flexibility in the design of the device by employing the directionality of the focused acoustic signal. The voltage of the electrical signal output from receiver 5B can then be used to activate or deactivate the positive feedback device upon performance of a specific desired movement by the user of the system.

For example, the transmitted acoustic signal from an extended fingertip may not be received by a receiver 5B positioned at the palm or finger base region of glove 3, when the user's fingers are extended substantially parallel to the palm. In this manner, finger extension activates the positive feedback system when the desired signal is not received by receiver 5B. The strength of the acoustic signal may be adjusted in order to increase the sensitivity of the system 200. By increasing the strength of acoustic signal, it may be possible to project a signal over greater distances.

To enhance accuracy, receivers 5B may be highly sensitive so as to reliably receive the transmitted signal. In an exemplary embodiment, receiver 5B may detect an electrical signal of at least 1 mV. In an exemplary embodiment, receiver 5B may be capable of detecting an acoustic signal when it is positioned at an angle of less than or equal to about 30° relative to the direction of transmission of transmitter 5A, preferably, at an angle of less than or equal to about 45° relative to the direction of transmission of transmitter 5A, and more preferably, at an angle of less than or equal to about 90° relative to the direction of transmission of transmitter. Upon receipt of an acoustic signal, receiver 5B generates an electrical signal having a voltage corresponding to the amplitude of the received acoustic signal.

In another exemplary embodiment, the sensors 5A, 5B may determine the degree of finger extension by measuring how long it takes for receiver 5B to receive a signal from transmitter 5A. Microcontroller 9 may function to track the elapsed time between signal transmittance and reception. The longer it takes to receive the signal, the greater the finger extension. The degree of finger extension may also be determined from the phase shift in the received signal. Microcontroller 9 may be used to compare the phase of the signal as it is generated by transmitter 5A with the phase of the signal when received by receiver 5B.

Optionally, system 1 may further include one or more conditioners 21 and/or amplifiers 23 to process the electrical signal produced by receiver 5B. Conditioner 21 may transform an alternating current signal produced by receiver 5B to a direct current signal, and amplifier 23 may increase the generated electrical signal to enhance sensitivity. When the electrical signal is amplified, signal interference is thereby minimized.

Physical therapy system 1 further includes a positive feedback system 7. An exemplary positive feedback system may have one or more microcontrollers 9, one or more counters 11, one or more positive feedback devices 13 and one or more power sources 15. Microcontroller 9 may be any computer chip capable of processing signals produced by receiver 5B and activating positive feedback device 13 when a precondition is met. For example, microcontroller 9 may be programmed to activate positive feedback device 13 when the voltage received from receiver 5B is less than or exceeds one or more preset threshold levels. In one embodiment, microcontroller 9 may operate as a simple on-off switch that activates positive feedback device 13. Alternatively, microcontroller 9 may be programmed with a plurality of thresholds so that microcontroller 9 may direct positive feedback device 13 to produce different responses corresponding to the voltage generated by receiver 5B.

For example, in one embodiment receiver 5B may generate gradually lower voltage electrical signals over time indicating increased finger extension, an audio response from positive feedback device 13 may become louder, longer, higher in pitch, produce different sounds or combinations thereof; a visual display may become brighter, produce different colors or images or combinations thereof; and/or a vibration device may produce different vibration sequences, vibrate longer, vibrate with greater intensity or combinations thereof to indicate the increased finger extension to the user. In this manner, the user may obtain a clear indication of when an exercise is achieving the desired result and when it is not.

Optionally, positive feedback system 7 may include a potentiometer 25 that is operatively associated with microcontroller 9 to establish and adjust the one or more preset threshold voltages. Potentiometer 25 may have a user friendly interface, such as a dial that can be turned and set to various positions that correspond to different degrees of finger extension. Potentiometer 25 may generate a positive or negative voltage, such as about −15V to about +15V.

One or more counters 11 may be operatively associated with microcontroller 9 and/or potentiometer 25 to record and monitor a user's progress over time. When microcontroller 9 determines that the voltage of an electrical signal produced by receiver 5B is less than a preset threshold, microcontroller 9 and/or potentiometer 25 may activate counter 11 to record a successful performance of finger extension. Additional counters 11 may be added to record different degrees of finger extension and/or unsuccessful finger extension attempts. In an exemplary embodiment, counter 11 may have an accuracy of about ±1%.

Positive feedback device 13 may be operatively associated with microcontroller 9 and/or counter 11 in order to provide an indicator to the user when finger extension has been properly achieved. Positive feedback device 13 may be any device suitable for providing a visceral response, preferably a multimodal response. Exemplary devices may include lighting elements, such as LEDs, audio generators and speakers, vibrational mechanisms, electrical shock mechanisms or combinations thereof. In an exemplary embodiment, positive feedback device 13 is capable of producing a wide range of responses so a user receives different, increased or enhanced responses as the user increases and/or more accurately executes proper finger extension. The stimulus may be frequently changed to avoid habituation. In an exemplary embodiment, positive feedback device 13 may be a television or computer that provides visual and audio stimuli. The television or computer may be used to record and/or display a user's performance over time and/or the data produced by a user's finger extension in real time. Additionally, in an exemplary embodiment, positive feedback device 13 may be an audio video device capable of recording and/or playing a prerecorded message, such as an encouraging or positive message delivered by a user's family member.

Any suitable power source 15 may be used to operate positive feedback system 7. In one embodiment, power may be obtained by plugging positive feedback system 7 into an outlet or other grid-based power source. Alternatively, power source 15 may be a portable device, such as a battery. In an exemplary embodiment, power source 15 maybe connected to microcontroller 9 and/or potentiometer 25.

In an exemplary embodiment, positive feedback system 7 may be externally connected to and separate from glove 3. Alternatively, positive feedback system 7 may be a small, portable device that may be positioned on a surface of, or within a cavity of, glove 3. Positive feedback system 7 and glove 3 may be operatively connected by wires or may be connected via any suitable conventional wireless system.

To operate physical therapy system 1 of the present invention, glove 3 may be placed on the hand of an intended wearer, including infants, children or adults. In an exemplary embodiment, glove 3 is placed on the hand of an individual affected with hemiplegic cerebral palsy. System 1 may be particularly suited to assist hemiplegic children, preferably children about 3 months old to about 5 years old, more preferably, children about 7 to about 25 months old or children about 3-7 months old.

As shown in FIGS. 2(a)-2(c), system 1 measures finger angle by analyzing the amount of acoustic signal projected from transmitter 5A that is received by receiver 5B. In the initial clenched orientation shown in FIG. 2(a), positive feedback system 7 is turned off when the acoustic signal from transmitter 5A is received by receiver 5B. Positive feedback system 7 activates when a user first begins to unclench his hand and extend his fingers, as shown in FIG. 2(b). As the fingers slowly extend, the received signal strength decreases because the receiver 5B moves gradually out of alignment with transmitter 5A. Upon full extension, receiver 5B should preferably receive little or no acoustic signal to simplify determination of an ideal finger extension from the voltage output by receiver 5B. Receiver 5B subsequently generates an electrical signal, the voltage of which may correspond to the amplitude of the received acoustic signal. Optionally, the electrical signal may be conditioned and amplified to enhance sensitivity.

The electrical signal is then processed by microcontroller 9, which may compare it to one or more preset voltage thresholds. In an exemplary embodiment, the voltage threshold may be adjusted, for example, by using the dial of the potentiometer 25. Adjustment of voltage thresholds may be useful for varying the amount of movement required to activate positive feedback system 7 to allow for flexibility in the rehabilitation method. When the voltage is lower than the preset threshold, microcontroller 9 activates counter 11 to record a successful finger extension. Additionally, microcontroller 9 activates positive feedback device 13 to generate a response to provide positive reinforcement to the user. In an exemplary embodiment, music will play and an LED will light up in interesting patterns when the fingers are properly extended. Guided by positive feedback, system 1 trains the user to accurately and properly extend and retract his fingers.

FIG. 3 shows an exemplary embodiment of a second physical therapy system 100 that may be used to teach clapping. The system may be constructed as a pair of gloves 103 having one or more sensors 105 suitable for transmitting and receiving acoustic signals and a positive feedback system 107. Gloves 103 may have the same size, shape and configuration and may be constructed from the same material as that of glove 3.

A first glove 103A may include one or more transmitters 105A, and a second glove 103B may have one or more receivers 105B suitable for receiving the acoustic signal transmitted by transmitter 105A. Sensors 105 may be positioned on one or more surfaces of, and/or in one or more interior cavities of glove 103 in order to effectively teach the user to clap. Exemplary positions for sensors 105 include any region of glove 103 housing the finger and/or palm when glove 103 is worn by an intended wearer. Preferably, sensors 105 are located on the portion of gloves 103 which covers the palm of the hand when worn by an intended wearer. In an exemplary embodiment, the placement of transmitter 105A on first glove 103A may correspond to the placement of receiver 105B on second glove 103B. For example, to provide gradated positive feedback to indicate when the hands are aligned closer to the ideal alignment for clapping. Transmitter 105A may also cover the entire palm facing surface of first glove 103A, and receiver 105B may cover the entire palm facing surface of second glove 103B if the goal is, for example, to encourage contact between the palm surfaces irrespective of alignment.

One or more pairs of sensors 106, 108 may also be positioned on gloves 103. For example, first glove 103A may include one or more transmitters 106A and one or more receivers 108B while second glove 103B may include one or more transmitters 108A and one or more receivers 106B. Alternatively, first glove 103A may include two or more transmitters 106A, 108A while second glove 103B may include two or more receivers 106B, 108B. In an exemplary embodiment, each transmitter 106A, 108A may be operatively associated with each receiver 106B, 108B of the system. Alternatively, each receiver 106B, 108B may uniquely correspond to only a specific transmitter 106A, 108A. In an exemplary embodiment, the sensors 106, 108 may operate within acceptable piezoelectric resonance frequencies. Alternatively, they may operate at lower or higher frequencies. A specific pair of sensors, i.e. 106A and 106B, may be capable of distinguishing signals from other sensors transmitting at different frequencies.

In another embodiment, sensors 105 may be positioned on or within first glove 103A in the same manner as sensors 5A, 5B are positioned on or within glove 3; a set of corresponding sensors 105 may be positioned on or within second glove 103B in a similar manner For example, one or more receivers 105B may be positioned at the palm of first glove 103A while one or more transmitters 105A may be positioned at the fingers of first glove 103A in order to train a user to clap with fingers in the extended position. In an exemplary embodiment, one or more transmitters 105A may be positioned at each finger of first glove 103A with one or more receivers 105B positioned at the palm. Correspondingly, one or more operatively associated receivers 105B may be positioned at the fingers of second glove 103B with one or more transmitters 105A positioned at the palm of that glove.

Sensors 105, transmitter 105A and receiver 105B may be the same as sensors 5, transmitter 5A and receiver 5B. In an exemplary embodiment, receiver 105B of second glove 103B only detects an acoustic signal when it is positioned about 2 cm to about 3 cm from transmitter 105A, preferably less than about 2 cm. Upon detecting an acoustic signal, receiver 105B subsequently produces an electrical signal corresponding to the amplitude of the detected ultrasonic signal.

In an alternative embodiment, sensor 105, transmitter 105A and receiver 105B may be a pressure sensors or magnetic inductance sensors suitable for detecting motion of both of the user's hands. In an exemplary embodiment, the pressure sensor may be a piezoelectric sensor that measures the applied pressure when contact is made between first glove 103A and second glove 103B. The pressure sensor may generate a voltage in a similar manner as the ultrasonic signal sensors. With respect to the magnetic induction sensor, first glove 103A may include a magnetic field generator and second glove 103B may include a receiver for receiving the generated magnetic field. The receiver may generate a voltage in a similar manner as the ultrasonic signal sensors. In an exemplary embodiment, the pressure and magnetic sensors may have the same detection sensitivity and detection range as that of the ultrasonic signal sensors.

Physical therapy system 100 may further include a positive feedback system 107 operatively associated with gloves 103. Positive feedback system 107 may include one or more microcontrollers 109, one or more counters 111, one or more positive feedback devices 113 and one or more power sources 115, one or more optional conditioners 121, one or more optional amplifiers 123 and one or more optional potentiometers 125 having the same structure and configuration as that of positive feedback system 7 of physical therapy system 1.

In an exemplary embodiment, an electrical conduit may connect gloves 103 to positive feedback system 107 so that positive feedback system 107 is externally connected to and separate from gloves 103. Alternatively, positive feedback system 107 may be positioned on one or within one of or both of gloves 103.

To operate physical therapy system 100, gloves 103 may be placed on the hands of any user, including infants, children or adults. In an exemplary embodiment, gloves 103 are placed on the hands of an individual affected with hemiplegic cerebral palsy.

As shown in FIG. 4, system 100 measures the distance between gloves 103 by analyzing the amplitude of acoustic signal from transmitter 105A of first glove 103A that is received by receiver 5B of second glove 103B. System 100 activates when a user first begins to draw his hands together. In an initial position wherein gloves 103 are separated, the voltage generated by receiver 105B responsive to the received signal from transmitter 105A is below a predetermined threshold voltage. Receiver 105B moves into closer proximity to transmitter 105A, and remains in alignment with the transmitted signal from transmitter 105A, the output voltage from receiver 105B will increase in proportion to an increase in the amplitude of the received signal. Receiver 105B should receive the maximum signal strength when gloves 103 directly contact one another in a manner whereby transmitter 105A and receiver 105B are aligned. Receiver 105B subsequently generates electrical signals, the voltage of which corresponds to the amplitude of the detected acoustic signals. Optionally, the electrical signal may be conditioned and amplified to enhance detection sensitivity. The electrical signal is then processed by microcontroller 109, which compares it to one or more preset voltage thresholds. In an exemplary embodiment, the voltage threshold may be adjusted using the dial of the potentiometer 125. When the voltage exceeds the preset threshold, microcontroller 109 may activate counter 111 to record a successful clap. Additionally, microcontroller 9 may activates positive feedback device 113 to generate a response to provide positive reinforcement to the user. In an exemplary embodiment, music will play and an LED will light up in interesting patterns when clapping is properly performed. In another exemplary embodiment, when the user claps, positive feedback device 113 will be activated based on a baseline assessment of the distance to which the user can already hold both hands. In this manner, gradual improvements from a baseline toward a successful outcome can be rewarded by positive feedback device 113 to thereby provide the ability to train the user in a gradual, stepwise manner, rather than an all-or-nothing scenario where the user is only rewarded for a completely successful outcome. Using positive feedback, system 100 trains the user to clap.

An alternative embodiment of system 100 may employ only one glove 103A having one or more proximity sensors. The proximity sensor may be a pressure sensor or an acoustic sensor capable of detecting the proximity of a glove 103A relative to the ungloved hand. In these embodiments, glove 103A is preferably placed on a debilitated hand.

When the proximity sensor is a pressure sensor, positive feedback may be provided when the force generated by contact between glove 103A and the ungloved hand is registered by the sensor. The pressure sensor then generates an electric signal that corresponds to the force produced by the aforementioned contact.

In another embodiment, glove 103A may include both an acoustic transmitter 105A and an acoustic receiver 105B. When glove 103A is brought into contact with the ungloved hand, acoustic signals produced by transmitter 105A are reflected off of the ungloved hand to receiver 105B. The remainder of the system then operates as described above, except that the system is calibrated for voltages corresponding to the amplitude of the reflected signal received by receiver 105B.

FIGS. 5(a)-5(b) show an exemplary embodiment of a third physical therapy system 200 for training arm extension. The system may be constructed as a glove 203 having a sensor 205 suitable for transmitting or receiving acoustic signals, and an object 204 spaced apart from glove 203 having a corresponding sensor 205 suitable for transmitting or receiving acoustic signals and a positive feedback system 207. Glove 203 may have the same size, shape and configuration and may be constructed from the same material as that of glove 3.

Object 204 may have any size, shape or configuration and may be constructed from any suitable material. In an exemplary embodiment, object 204 may be a movably positioned; preferably, the position of object 204 may be vertically and horizontally adjusted. For example, object 204 may include one or more mounting structures, such as a hook, aperture, catch, adhesive, suction cup, stand or combinations thereof that enables object 204 to stand on a flat surface, such as a table top, and/or be removably mounted to a vertical surface such as a wall, door or mirror. In an exemplary embodiment, object 204 may have a facade that is user friendly and attractive to young children. Exemplary embodiments of object 204 may include a movably positioned activity board or panel. Glove 203 may include one or more transmitters 205A, and object 204 may have one or more receivers 205B suitable for receiving acoustic signals transmitted by transmitter 205A. Sensors 205 may be strategically positioned on or within object 204 and on or within a palm-facing surface of glove 203 in order to facilitate teaching of arm extension. In an exemplary embodiment, one or more transmitters 205A may be positioned a region housing the fingers or palm of glove 203, and one or more corresponding receivers 205B may be positioned on a surface of or within object 204. for the purpose of teaching arm extension, transmitter 205A may optionally cover the entire palm-facing surface of glove 203, and receiver 205B may optionally cover the entire surface of object 204.

As shown in FIG. 5(a), object 204 may include a plurality of receivers 205B positioned at different locations along object 204 to train different degrees of arm extension. For example, receivers 205B may be positioned in a linear vertical, horizontal or diagonal line wherein receivers 205B are each separated by a distance of at least about 2 cm to about 3 cm, preferably less than about 2 cm. Alternatively, receivers 205B may be arranged in a geometric configuration, such as a circle, square, triangle, etc. Receivers 205B may also be arranged in rows and columns or in concentric geometric patterns, such as concentric circles, that fill a surface of object 204. In an exemplary embodiment, receivers 205B may be removably attached to object 204 enabling adjustable positioning of receivers 205B. Receivers 205B may be removably attached to object 204 using any conventional fastener, such as hook and loop structures, adhesive, claps, latches, snaps, string or combinations thereof, that may be attached to a surface of receiver 205B and/or object 204. Using patterned arrays of receivers 205B allows more specific feedback to the user to encourage, for example, arm extension in a horizontal position, or at an upward, downward or sideward angle, if desired. In this manner, the arm extension exercises can be varied for the purpose of tailoring the exercise to a specific therapeutic regimen or simply to provide a more interesting and varied exercise program.

Physical therapy system 200 may further include a positive feedback system 207 having the same components, arrangement, function and structure such as those discussed above.

In an exemplary embodiment, an electrical conduit may connect glove 203 and object 204 to positive feedback system 207 so that positive feedback system 207 is externally connected to and separate from glove 203 and object 205. Alternatively, positive feedback system 107 may be positioned on one or within one of or both of gloves 103. Alternatively, positive feedback system 207 may be positioned on or within glove 203 or object 204. Preferably, positive feedback system 207 may be positioned on or within object 204. For example, object 204 may include LED lights, display screen, audio generator and speakers or combinations thereof.

To ensure that a user activates the system by extending his arm rather than by walking glove 203 towards object 204, the user may be temporarily restrained in a chair and placed before object 204. Alternatively, system 200 may further include a pressure activated seat cushion 217 including one or more pressure sensors 219 connected to positive feedback system 207. When the user sits on cushion 217, system 200 may operate as normal. When, however, cushion 217 does not detect a user's weight, system 200 is deactivated to thereby ensure that the user remains in the chair during the exercise. In an exemplary embodiment, the pressure threshold of cushion 217 maybe adjusted depending upon the weight of the user.

To operate physical therapy system 200, glove 203 may be placed on the disabled hand of any user, including infants, children or adults. In an exemplary embodiment, glove 203 is placed on the disabled hands of an individual affected with hemiplegic cerebral palsy. Object 204 may then be positioned on or mounted to a surface. The user may then be seated on a cushion 217 in front of object 204. The chair should be positioned so that the user may be within arm's length of object 204.

As shown in FIG. 6, system 200 activates when a user first begins to draw glove 203 towards object 204. In an initial position wherein glove 203 and object 204 are separated, the output voltage of receiver 205B of object 204 is below a threshold level. Output voltage of receiver 205B will increase as receiver 205B is moved into closer proximity to transmitter 205A. Receiver 205B should preferably receive the maximum acoustic signal when glove 203 directly contacts object 204. System 200 operates in a similar manner as system 100.

For purposes of the present invention, the position of the one or more transmitters 5A, 105A, 106A, 108A, 205A and corresponding one or more receivers 5B, 105B, 106B, 108B, 205B as referred to in any of the aforementioned embodiments may be exchanged with one another. For example, in physical therapy system 1, one or more transmitters 5A may be positioned at the fingertips of glove 3 with one or more corresponding receivers 5B positioned at the palm of glove 3. Alternatively, one or more transmitters 5A may be positioned at the palm of glove 3 with one or more corresponding receivers 5B positioned at the fingertips of glove 3. Similarly, in physical therapy system 200, one or more transmitters 203A may be positioned on or within glove 203 with one or more corresponding receivers 205B positioned on or within object 207. Alternatively, one or more transmitters 205A may be positioned on or within object 207 with one or more corresponding receivers 205B may be positioned on or within glove 203.

The physical therapy systems 1, 100 and 200 of the present invention offer a number of advantages over rehabilitation devices of the prior art. The invention is particularly advantageous in that a physical therapist need not be present in order to perform the rehabilitation exercises, thereby reducing healthcare costs and increasing the number of patients that can be effectively treated. Because the system may be compact and portable, it enables treatment within the privacy of the user's own homes. Another advantageous feature of the present invention is the ability to treat children or disabled individuals that would otherwise be agitated by, or children who are too young to be eligible for, conventional constraint-induced movement therapy. Additionally, the system can be easily adapted to provide quantitative data which can be tracked over time to monitor a user's progress.

Having described the preferred embodiments of the invention which are intended to be illustrative and not limiting, it is noted that modifications and variations can be made by persons skilled in the art in light of the above teachings. It is therefore to be understood that changes may be made in the particular embodiments of the invention disclosed which are within the scope and spirit of the invention as outlined by the appended claims. Having thus described the invention with the details and particularity required by the patent laws, the intended scope of protection is set forth in the appended claims.

1. A physical therapy system comprising:
a first object;
a first piezoelectric sensor associated with said object and which is capable of transmitting an acoustic signal;
a second piezoelectric sensor associated with said object and which is capable of receiving said acoustic signal from said first piezoelectric sensor and converting said acoustic signal to an electrical signal; and
a positive feedback system operably associated with said second piezoelectric sensor for activation responsive to said electrical signal.

2. The system of claim 1, wherein said first and second piezoelectric sensors are polyvinylidene fluoride sensors.

3. The system of claim 1, wherein said first or second piezoelectric sensor has a detection sensitivity of at least 1 mV.

4. The system of claim 1, wherein said object is a glove and said first piezoelectric sensor is positioned on or within a distal region of said glove corresponding to the location of an individual's fingertips when said glove is worn by an intended wearer.

5. The system of claim 1, wherein said object is a glove and said first piezoelectric sensor is positioned on or within a distal region of said glove corresponding to a location of a first fingertip when said glove is worn by an intended wearer and wherein said system further comprises one or more additional piezoelectric sensors positioned on or within a distal region of said first glove corresponding to a location of one or more additional fingertips, when said glove is worn by an intended wearer.

6. The system of claim 4, wherein said second piezoelectric sensor is positioned on or within a proximal region of said glove corresponding to the location of an individual's palm or palm base when said glove is worn by an intended wearer.

7. The system of claim 4, wherein said second piezoelectric sensor is positioned on or within a middle region of said glove corresponding to the location of an individual's finger base when said glove is worn by an intended wearer.

8. The system of claim 1, further comprising a counter capable of tracking the progress of a user by counting a number of successful iterations of a desired movement of a user.

9. The system of claim 1, wherein said positive feedback system further includes a potentiometer for adjusting a threshold value of the electrical signal from said second piezoelectric sensor which activates one or more features of said positive feedback system.

10. A physical therapy system comprising:
a first glove;
a first piezoelectric sensor associated with said first glove;
an object;
a second piezoelectric sensor associated with said object, wherein one said piezoelectric sensor is capable of transmitting an acoustic signal and another said piezoelectric sensor is capable of receiving said transmitted acoustic signal from said piezoelectric sensor and converting said acoustic signal to an electrical signal; and
a positive feedback system operably associated with said second piezoelectric sensor which converts the acoustic signal to the electric signal, for activation responsive to said electrical signal.

11. The system of claim 10, wherein said object is selected from the group consisting of a panel, activity board, and a glove.

12. The system of claim 10, wherein said first and second piezoelectric sensors are polyvinylidene fluoride sensors.

13. The system of claim 10, wherein said system further includes a seat cushion comprising at least one pressure sensor operatively connected to said positive feedback system in a manner whereby exertion of pressure on said pressure sensor activates said positive feedback system.

14. The system of claim 10, wherein said object comprises a plurality of piezoelectric sensors adapted to receive acoustic signals and convert said acoustic signals to electrical signals.

15. The system of claim 11, wherein said object is a second glove and wherein said second piezoelectric sensor is positioned on or within a finger region of said second glove.

16. The system of claim 10, further comprising a counter capable of tracking the progress of a user by counting a number of successful iterations of a desired movement of a user.

17. A physical therapy method comprising the steps of:
providing a physical therapy system comprising
a glove; a first piezoelectric sensor associated with said glove and which is capable of transmitting an acoustic signal;
a second piezoelectric sensor associated with said glove and which is capable of receiving said acoustic signal from said first piezoelectric sensor; and
a positive feedback system operably associated with said second piezoelectric sensor for activation responsive to said electrical signal;
placing said glove on a hand of an individual;
extending a finger of said hand so that said finger is substantially parallel to a palm of said hand whereby said positive feedback system is activated.

18. A physical therapy method comprising the steps of:
providing a physical therapy system comprising:
a first glove;
a first piezoelectric sensor associated with said first glove;
a second glove;
a second piezoelectric sensor associated with said second glove, wherein one of said piezoelectric sensors transmits an acoustic signal and the other of said piezoelectric sensors receives the transmitted acoustic signal and converts it to an electrical signal; and
a positive feedback system operatively associated with the piezoelectric sensor that converts the acoustic signal to the electrical signal for activation responsive to said electrical signal;
placing said first and second gloves on the hands of an intended wearer; and
activating said positive feedback system to produce positive feedback responsive to said electrical signal from said piezoelectric sensor.

19. A physical therapy method as claimed in claim 18, wherein said positive feedback system activates when a voltage of said electrical signal exceeds or falls below a predetermined threshold voltage.

20. A physical therapy method as claimed in claim 19, wherein said positive feedback system is responsive to a plurality of different predetermined threshold voltages to provide a plurality of different feedbacks, each said feedback being provided when said electrical signal exceeds or falls below one of said plurality of different predetermined threshold voltages.
